(19) United States
(12) Patent Application Publication
(10) Pub. No.: US 2007/0299423 A1
(43) Pub. Date: Dec. 27, 2007

(54) WIRE STABILIZATION

(75) Inventors: Lloyd Jones, Auckland (NZ); Timothy Stivland, Los Altos, CA (US); Jayson Delos Santos, Pinole, CA (US); Herbert Mendoza, So. San Francisco, CA (US); Ari Ryan, Mountain View, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/762,600

(22) Filed: Jun. 13, 2007

Related U.S. Application Data
(60) Provisional application No. 60/805,820, filed on Jun. 26, 2006.

(51) Int. Cl. A61M 25/00

(52) U.S. Cl. 604/523

(57) ABSTRACT

Devices constrain a distal portion or region of a wire, such as a guidewire or a filterwire, from movement at or near a proximal portion or region of the wire. A magnetic field may be used to constrain a wire passing through a hub. A clamping apparatus may include first and second clamps that, when used in combination, restrain wire movement while advancing an exchange device. A guide catheter may include an anchor movable between an open configuration and a closed configuration.

This application claims the benefit of U.S. Provisional Application No. 60/805,820, entitled “WIRE STABILIZATION,” filed Jun. 26, 2006, the entirety of which is herein incorporated by reference.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences because the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices, termed embolic protection devices, have been developed to filter out this debris.

A wide variety of medical devices have been developed for medical use, for example, intravascular use. Of the known devices, each has certain advantages and disadvantages. There is an ongoing need to provide alternative devices. In particular, there is an ongoing need for devices that isolate a distal portion of a wire from movement along a proximal portion of the wire.

### SUMMARY

The invention pertains generally to devices that permit a distal portion or region of a wire, such as a guidewire or a filterwire, from movement at or near a proximal portion or region of the wire.

Accordingly, an illustrative but non-limiting example of the present invention may be found in a hub assembly that includes a hub body that defines a hub lumen extending through the hub body. A magnetic column may be disposed exterior to the hub lumen, and a wire may be disposed within the hub lumen.

Another illustrative but non-limiting example of the present invention may be found in a hub that includes a hub body defining a hub lumen. A magnetic column may be disposed at least partially about the hub lumen, and an introducer sheath may be removably disposed within the hub lumen. The introducer sheath may be configured to reduce a magnetic field within at least a portion of the hub lumen.

Another illustrative but non-limiting example of the present invention may be found in a hub assembly that has a hub body having an interior and an exterior. The interior of the hub body may define a hub lumen. A wire may be disposed within the hub lumen and a magnetic column may be disposed about the hub body exterior.

Another illustrative but non-limiting example of the present invention may be found in a clamping apparatus. The clamping apparatus includes an elongate body that has a distal end and a proximal end. The distal end may be adapted to be secured to a medical hub. A first clamp and a second clamp are both secured to the elongate body such that the second clamp is spaced apart from the first clamp.

Another illustrative but non-limiting example of the present invention may be found in a method of advancing a medical device including a monorail section over a wire. A wire may be advanced through a medical apparatus that includes a clamping apparatus having a first clamp and a second clamp spaced apart from the first clamp, the clamping apparatus extending proximally from the medical apparatus.

The first clamp may be secured to the wire, and the medical device monorail section may be advanced over the wire such that the monorail section is disposed between the first clamp and the second clamp. The second clamp may be secured to the wire, and the first clamp may be released. The medical device may then be advanced further over the wire.

Another illustrative but non-limiting example of the present invention may be found in an assembly that includes a guide catheter having a distal region, a proximal region and a lumen that extends from the distal region to the proximal region. An elongate shaft may be positioned such that a proximal portion of the elongate shaft is disposed within the lumen while a distal portion of the elongate shaft extends distally beyond the guide catheter. An embolic protection device may be disposed on the distal portion of the elongate shaft. An anchor may be disposed within the distal region of the guide catheter. The anchor may have an activated configuration in which the anchor extends considerably into the lumen and an inactivated configuration in which the anchor does not extend considerably into the lumen.

The above summary of the present invention is not intended to describe each disclosed embodiment or every implementation of the present invention. The Figures, Detailed Description and Examples which follow more particularly exemplify these embodiments.

### BRIEF DESCRIPTION OF THE FIGURES

The invention may be more completely understood in consideration of the following detailed description of various embodiments of the invention in connection with the accompanying drawings, in which:

FIG. 1 is a view of a hub in accordance with an illustrative but non-limiting example of the invention;

FIG. 2 is a view of an assembly in accordance with an illustrative but non-limiting example of the invention;

FIG. 3 is a view of an introducer sheath in accordance with an illustrative but non-limiting example of the invention;

FIG. 4 is a view of an assembly in accordance with an illustrative but non-limiting example of the invention;

FIG. 5 is a view of a hub in accordance with an illustrative but non-limiting example of the invention;

FIG. 6A is a view of an external magnetic column in accordance with an illustrative but non-limiting example of the invention;

FIG. 6B is a view of an external magnetic column in accordance with an illustrative but non-limiting example of the invention;

FIG. 7 is a view of a hub in accordance with an illustrative but non-limiting example of the invention;

FIG. 8 is a view of a clamping apparatus in accordance with an illustrative but non-limiting example of the invention;

FIG. 9 is a view of an assembly in accordance with an illustrative but non-limiting example of the invention;

FIG. 10 is a view of an assembly in accordance with an illustrative but non-limiting example of the invention;

FIG. 11 is a view of an assembly in accordance with an illustrative but non-limiting example of the invention;

FIG. 12 is a view of an assembly in accordance with an illustrative but non-limiting example of the invention;

FIG. 13 is a view of an assembly in accordance with an illustrative but non-limiting example of the invention;

FIG. 14 is a view of an assembly in accordance with an illustrative but non-limiting example of the invention; and

FIG. 15 is a cross-section view taken along line 15-15 of FIG. 14.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative embodiments of the claimed invention.

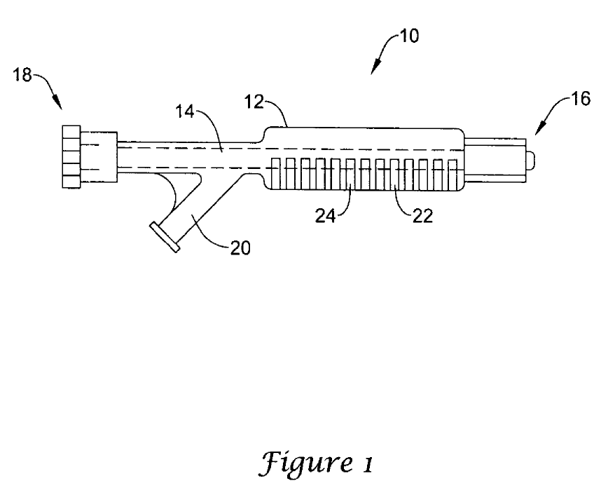

The invention pertains generally to devices that are adapted to reduce movement that may otherwise be transmitted to a distal region of an elongate shaft such as a guidewire or a filterwire. Movement reduction may be accomplished in a variety of manners. FIGS. 1 through 7 demonstrate an illustrative but non-limiting embodiment of the invention in which magnets are used to reduce or eliminate movement in a wire such as a guidewire or a filterwire. In this, movement may be defined as axial movement, radial movement, or a combination of both axial and radial movement.

FIG. 1 illustrates a hub 10 that includes a hub body 12 and a hub lumen 14 that extends through the hub body 12. The hub body 12 may be formed of any suitable polymeric material. In some instances, it may be desirable to be able to visualize the interior of the hub lumen 14 during preparation, to ensure that any fluid within the hub lumen 14 is free of air bubbles that could otherwise be detrimental to a patient. In such cases, the hub body 12 may be formed of a transparent or translucent polymeric material such as polycarbonate.

The hub 10 includes a distal end 16 that may, if desired, include a luer fitting that is adapted to be secured to any number of medical devices such as a guide catheter, and a proximal end 18. The proximal end 18 may be configured to permit access to the hub lumen 14 so that wires, catheters and the like may be advanced through the hub 10 and into a guide catheter that may, as noted, be secured to the distal end 16. In some instances, although not required, the proximal end 18 may include a rotating hemostatic valve. In some cases, as illustrated, the hub 10 may include a Y-adaptor 20 that can be used for injecting fluid and other uses as are known.

The hub 10 also includes a magnetic column 22. As will be discussed hereinafter, the magnetic column 22 may serve to stabilize or secure an elongate shaft such as a wire extending through the hub 10, in some instances, magnetic column 22 is positioned about or within the hub body 12 such that the magnetic column 22 at least partially surrounds the hub lumen 14. In some cases, the magnetic column 22 may extend at least about halfway around the hub lumen 14, or may even extend up to about three quarters of the way around the hub lumen 14. To put it another way, the magnetic column may, in some instances, extend from about 180 degrees to about 270 degrees around the hub lumen 14.

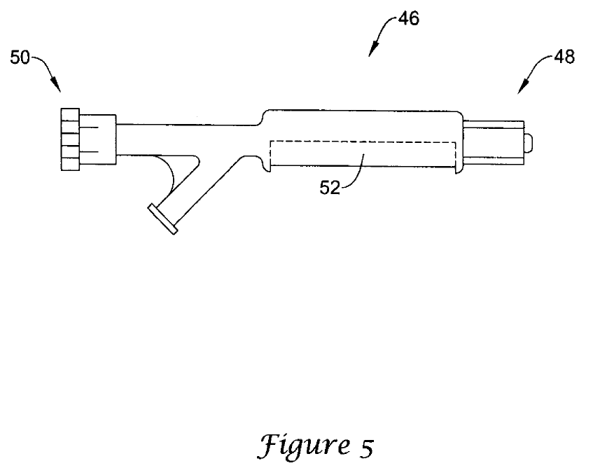

The magnetic column 22 may be formed from a single magnet, or may, as illustrated, include a number of individual magnets 24. In some cases, the magnet or magnets forming the magnetic column 22 may be molded within the hub body 12. In other instances, as will be discussed in greater detail hereinafter with respect to FIGS. 5 through 7, the magnet or magnets forming the magnetic column 22 may be positioned exterior to the hub body 12 and may in fact be removable. Any suitable magnetic material may be used to create the magnet or magnets used to form the magnetic column 22. In some instances, neodymium magnets may be useful.

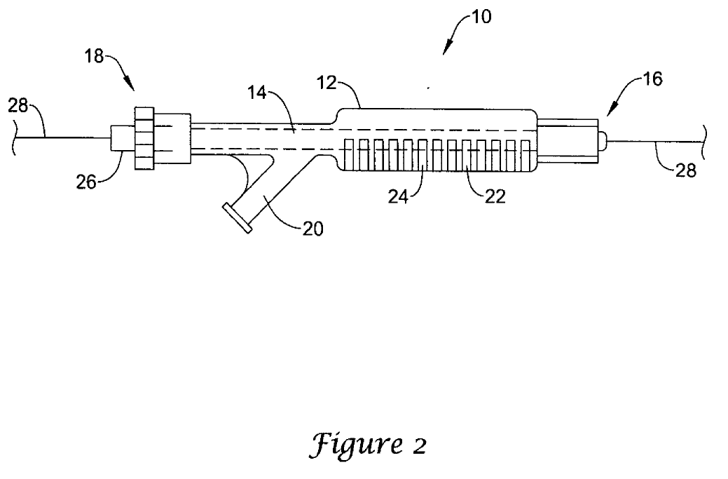

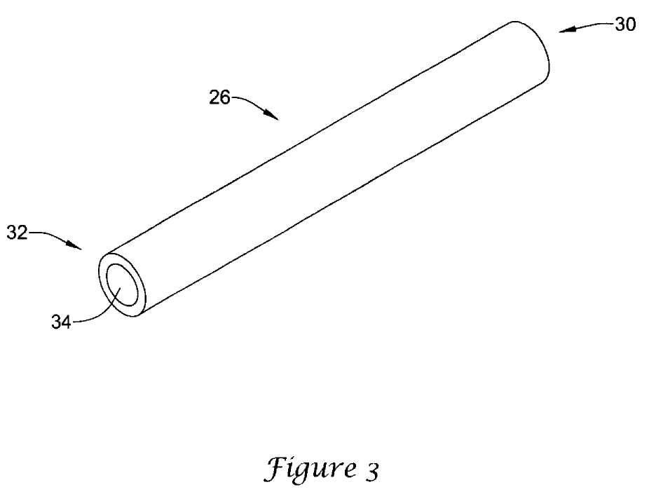

Turning now to FIG. 2, it can be seen that several additional structures are present. A sheath 26 is disposed within the hub lumen 14, and a wire 28 extends through the sheath 26. The wire 28 may be a guidewire or a filterwire. The sheath 26 is better seen in FIG. 3 as including a distal end 30, a proximal end 32 and a sheath lumen 34 extending between the distal end 30 and the proximal end 32. The sheath 26 may, as illustrated, have at least a largely cylindrical shape and may be sized to fit within the hub lumen 14. The sheath lumen 34 may be adapted to accommodate the wire 28, as well as any therapeutic or interventional medical device that may be advanced over the wire 28.

The sheath 26 may function to reduce the magnetic field that would otherwise permeate the hub lumen 14 as a result of the presence of the magnetic column 22. The sheath 26 does not, properly speaking, shield or stop magnetic field lines from entering the hub lumen 14 in the manner that lead stops X-rays, for example. Rather, the sheath 26 reduces the magnetic field within the hub lumen 14 by providing an alternate route for the magnetic field lines to follow.

Magnetic field lines emanating from a magnet travel between the north pole of the magnet and the south pole of the magnet. In doing so, however, magnetic field lines will follow the path of least resistance. Air, for example, has a magnetic permeability of about 1. Some materials, however, have magnetic permeability values that are much, much higher. Magnetic field lines emanating from the magnetic column 22 will follow a high magnetic permeability material, if it is present, rather than extending through the air. Thus, forming the sheath 26 of a high magnetic permeability material means that when the sheath 26 is disposed within the hub lumen 14, the magnetic field lines emanating from the magnetic column 22 will travel through the walls of the sheath 26, rather than through the air or fluid within the hub lumen 14.

A variety of high magnetic permeability materials are known. Examples include METGLAS®, which is a commercially available material containing largely cobalt, along with relatively minor amounts of nickel, iron, silicon and boron. This material has a magnetic permeability as high as about 1,000,000 and is available in thicknesses as little as 0.00065 inches (16 microns). Other suitable materials include alloys containing substantial amounts of nickel.

In order for the magnetic column 22 to stabilize or otherwise secure or immobilize the wire 28, at least a portion of the wire 28 may include or be formed from a material that responds to magnetic fields. For example, certain stainless steel alloys are known in the art as being responsive to magnetic fields. The wire 28 itself may be formed of a magnetically-responsive material, or may include an insert or even a coating of such a material.

In some cases, only a proximal portion, i.e. the portion disposed within the hub 10 may include a magnetically-responsive material. Other portions of the wire 28 may be formed of any metallic or polymeric material possessing desired strength characteristics. Examples include carbon fiber, liquid crystal polymer, or even a fiber-reinforced polymer or polymer blend.

It will be recognized, then, that as long as the sheath 26 is disposed within the hub lumen 14 such that wire 28 passes through the sheath 26, the wire 28 will be at least partially shielded from the magnetic field lines emanating from magnetic column 22. As a result, the wire 28 may be moved relative to the hub 10. If the sheath 26 has been removed, however, the magnetically-responsive portion or portions of the wire 28 will respond to the magnetic field generated by the magnetic column 22, and the wire 28 may be held motionless with respect to the hub 10.

As illustrated, the sheath 26 is disposed within the hub lumen 14. In some cases, the sheath 26 may be removed by pulling the sheath 26 proximally through the proximal end 18 of the hub 10. It will be recognized that the sheath 26 may be formed of a material that is thin enough to be easily torn. Thus, in some instances, it may be useful to remove the sheath 26 by pulling the sheath 26 proximally through Y-adaptor 20, tearing the sheath 26 away from the wire 28.

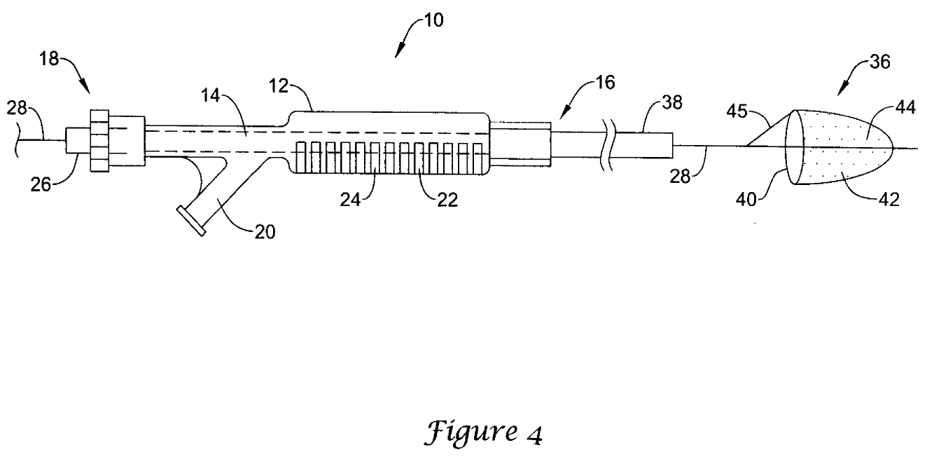

One useful application for the hub 10 can be seen in FIG. 4, in which an embolic protection device 36 is secured to the wire 28. If desired, a guide catheter 38 may be secured to the distal end 16 of the hub 10 and may extend distally to a position proximal to the embolic protection device 36. The guide catheter 38 may be formed of any suitable materials, as is known in the art.

The embolic protection device 36 may include a filter loop 40 and a filter membrane 42 that is secured to the filter loop 40. In some cases, if desired, filter membrane 42 may be drilled (for example, formed by known laser techniques) or otherwise manufactured to include a plurality of openings 44. These holes or openings 44 can be sized to allow blood flow therethrough but restrict flow of debris or emboli floating in the body lumen or cavity.

In general, embolic protection device 36 may be adapted to operate between a first generally collapsed configuration and a second generally expanded configuration (as shown in FIG. 4, for example) for collecting debris in a body lumen. To this end, in at least some embodiments, filter loop 40 may be formed of or may include a “self-expanding” shape-memory material such as nickel-titanium alloy, which is capable of biasing embolic protection device 36 toward being in the second expanded configuration. Additionally, filter loop 40 may include a radiopaque material or include, for example, a radiopaque wire disposed about a portion thereof. Some further details regarding these and other suitable materials are provided below.

One or more struts 45 may extend between filter loop 40 and wire 28. Strut 45 may be secured at one end to the filter loop 40 and at the other end to the wire 28. Strut 45 may be secured at either end in any suitable manner, including soldering, laser welding, adhesives, or mechanically such as by wrapping one end of strut 45 several times about filter loop 40 and wrapping the other end of strut 45 several times about the wire 28. It will be recognized the configuration and even the number of struts 45 may be tailored and/or customized for a particular intervention.

In some cases, the wire 28, bearing the embolic protection device 36, may be extended through hub lumen 14 and through guide catheter 38 while the embolic protection device 36 is in its generally collapsed configuration. While extending the wire 28, it is contemplated that the sheath 26 remains within hub lumen 14 in order to at least partially reduce the magnetic field within the hub lumen 14 that would otherwise exist. As a result, the wire 28 may move relative to the hub 10.

Once the embolic protection device 36 has reached a desired location, it may be deployed by urging the embolic protection device 36 into its generally expanded configuration. Once the embolic protection device 36 has been deployed, the relative position of the wire 28, and hence the embolic protection device 36, may be stabilized by removing the sheath 26 from the hub lumen 14, as discussed previously with respect to FIG. 2. In some instances, it may be desirable to instead stabilize the position of the wire 28 prior to actually deploying the embolic protection device 36. To subsequently remove the wire 28, the sheath 26 may be reinserted into the hub lumen 14. Alternatively, it is contemplated that the hub 10 may be disconnected from the guide catheter 38, and the hub 10 and the wire 28 may be removed together.

As illustrated thus far, the magnetic column 22 has been shown as being molded into the hub body 12. The magnetic column 22, as discussed, may be a single magnet or a number of individual magnets. In some instances, however, a magnetic column may be disposed exterior to the hub body 12. FIGS. 5 through 7 provide illustrative but non-limiting examples of magnetic columns that are adapted to be deployed externally, as well as a hub adapted to accommodate such magnetic columns.

FIG. 5 shows a hub 46 having a distal end 48 and a proximal end 50. Details of the distal end 48 and the proximal end 50 may be similar to that of distal end 16 and proximal end 18, as discussed for example with respect to FIG. 1. However, the hub 46 includes a recessed portion 52 that is configured to accommodate an external magnetic column. The recessed portion 52 may be molded into the hub 46, or the recessed portion 52 may be formed by grinding or otherwise processing the hub 46. The recessed portion 52 may extend over a substantial length of the hub 46, if desired.

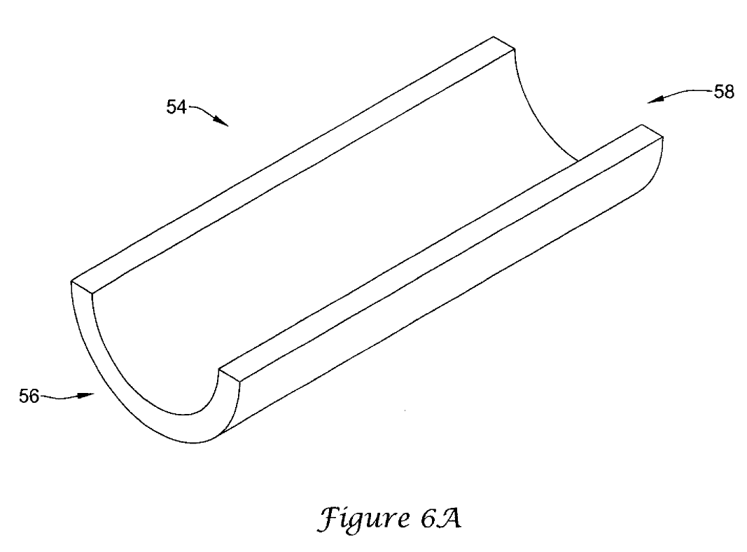

FIG. 6A shows a magnetic column 54 having a distal end 56 and a proximal end 58. It can be seen that the magnetic column 54 has a semi-circular profile and is configured to fit into the recessed portion 52 (FIG. 5). In some instances, the magnetic column 54 and the recessed portion 52 may be proportioned such that the magnetic column 54 snap-fits into the recessed portion 52. In some cases, it is contemplated that the magnetic column 54 may be held in place by an external structure. For example, the magnetic column 54 may be held in place by adhesive tape, if desired.

FIG. 6B shows a magnetic column 60 having a distal end 62 and a proximal end 64. As illustrated, the magnetic column 60 has a semi-circular profile. Unlike the magnetic column 54 shown in FIG. 6, however, the magnetic column 60 may extend more than 180 degrees, as the magnetic column 60 includes a hinge 66 that runs the length of the magnetic column 60, from distal end 62 to proximal end 64. In some instances, the magnetic column 60 may be opened along the hinge 66 prior to being inserted into recessed portion 52 (FIG. 5), and may then be closed. In some cases, the hinge 66 may be biased into a closed position. As a result, the magnetic column 60 may essentially hold itself in position within the recessed portion 52.

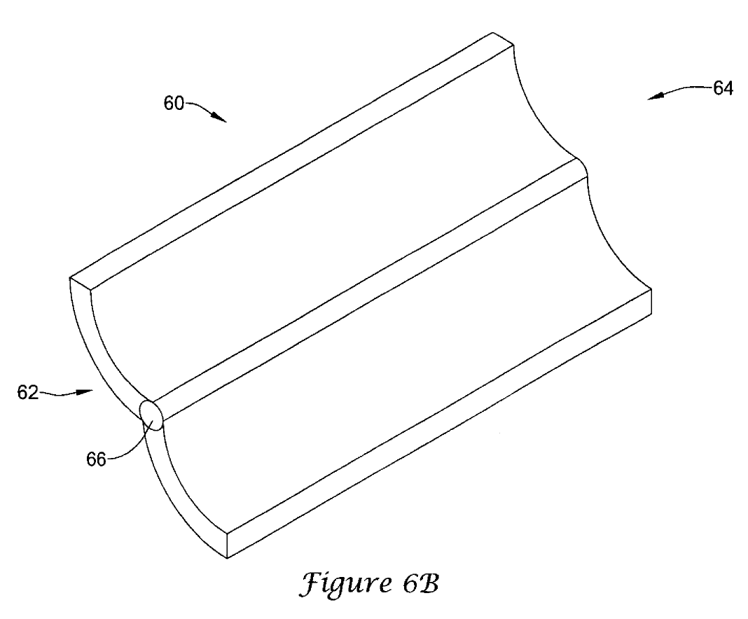

FIG. 7 shows a hub assembly 47 in which a hub 46 (FIG. 5) has been combined with a magnetic column 54 (FIG. 6A). As illustrated, it can be seen that the proximal end 58 is attached via a hinge 55 to the hub 46. In some instances, the magnetic column 54 may be configured to fit snugly about the recessed portion 52. In some cases, the recessed portion 52 may be excluded, and the magnetic column 54 may be configured to fit about the hub 46.

The hinge 55 may be any suitable hinge that permits the magnetic column 54 to be movable between a position in which the magnetic column 54 is at least partially removed from the hub 46 (as illustrated) and a position in which the magnetic column 54 is disposed about the hub 46. Thus, it can be seen that the magnetic column 54 may be easily moved between the illustrated position in which a wire may be freely moved and a position in which wire movement is restricted or prevented.

While not illustrated, it is also considered that a magnetic column 60 (FIG. 6B) could include a second hinge (not shown) that would permit the magnetic column 60 to be hingedly attached along a long side thereof to a hub 46 (FIG. 5). Not only would this permit the magnetic column 60 to be moved either into a wire movement position or a wire movement restriction position, but since the magnetic column 60 includes a longitudinal hinge 66 (FIG. 6B), this arrangement would permit use of a magnetic column 60 that extends further about the circumference of the hub 46.

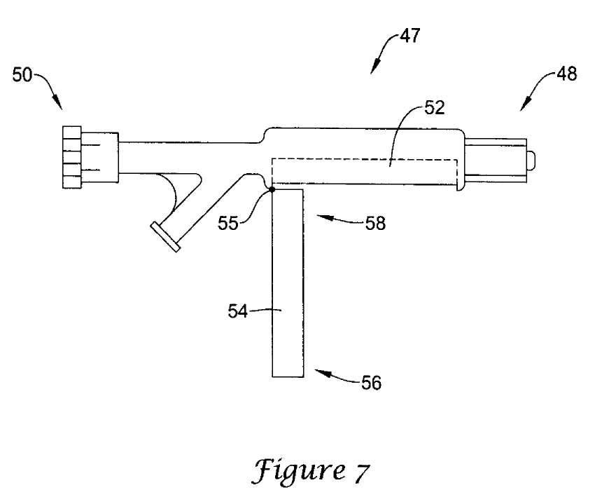

FIGS. 8 through 10 demonstrate an illustrative but non-limiting embodiment of the invention in which a clamping apparatus is used to reduce or eliminate movement in a wire such as a guidewire or a filterwire. As noted previously, movement may be defined as axial movement, radial movement, or a combination of both axial and radial movement.

FIG. 8 illustrates a clamping apparatus 68 that is configured to be attached to a medical device and to releasably secure a wire such as a guidewire or a filter wire so that additional devices may be advanced over a wire without transmitting undesired movement to the wire. The clamping apparatus 68 includes a body 70 that may be formed of any suitable metallic or polymeric material and having any appropriate dimensions. The body 70 includes an attachment section 72 and a clamping section 74.

The attachment section 72 may be adapted to secure the clamping apparatus 68 to a medical device such as a hub, a guide catheter, or the like. The attachment section 72 may include any suitable attachment mechanisms, such as an adhesive that permits securement to an appropriate medical device. In some instances, the attachment section 72 may provide mechanical attachment, such as a clamp, hook and loop securement, or the like, to an appropriate medical device.

The clamping section 74 includes a first clamp 76 and a second clamp 78. The first clamp 76 and the second clamp 78 may each be convertible between an open position in which an elongate medical device such as a catheter or other therapeutic device may fit through the clamp and a closed position in which a wire such as a guidewire or a filterwire is at least partially constrained from movement. The first clamp 76 and the second clamp 78 may each, independently, be either mechanically or electromagnetically activated.

The clamping apparatus 68 is configured to be used in conjunction with a catheter or similar device that includes a monorail section. In some instances, such a catheter may be referred to as a single operator exchange catheter or a rapid exchange catheter. Such catheters may have a relatively short guidewire lumen position at or near the distal end of the catheter. The guidewire lumen may be relatively short, such as, for example, about 10 to about 20 centimeters in length. This permits a physician or other professional to easily and quickly exchange one device for another over a single guidewire or filterwire.

The first clamp 76 and the second clamp 78 may, therefore, be positioned a distance apart that accommodates the monorail section of a catheter. In some instances, the first clamp 76 and the second clamp 78 may be fixedly secured to the body 70 and may be spaced apart an appropriate distance, as discussed. In some cases, it is contemplated that one of the first clamp 76 and the second clamp 78 may be fixedly secured to the body 70 while the other of the first clamp 76 and the second clamp 78 may be movably secured to the body 70. Thus, a physician or other profession may adjust the inter-clamp spacing in order to accommodate a particular exchange device.

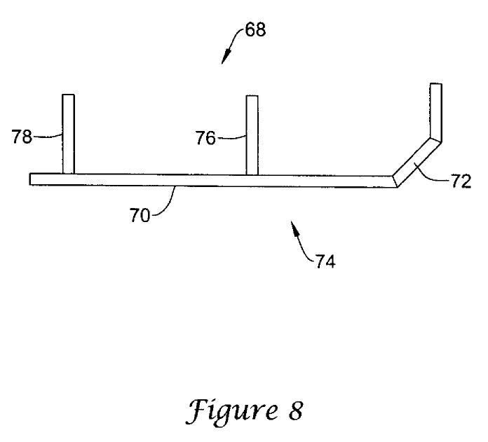

FIG. 9 illustrates the clamping apparatus 68 secured to a medical device. As illustrated, the clamping apparatus 68 is secured to a hub 80, but could instead be secured to any other appropriate device. The hub 80 includes a distal end 82 and a proximal end 84. Details of the distal end 82 and the proximal end 84 may be similar to that of distal end 16 and proximal end 18, as discussed for example with respect to FIG. 1. It can be seen that the attachment section 72 of the clamping apparatus 68 attaches to the hub 80 near the proximal end 84 thereof A wire 86 extends through the hub 80 and through the clamping apparatus 68.

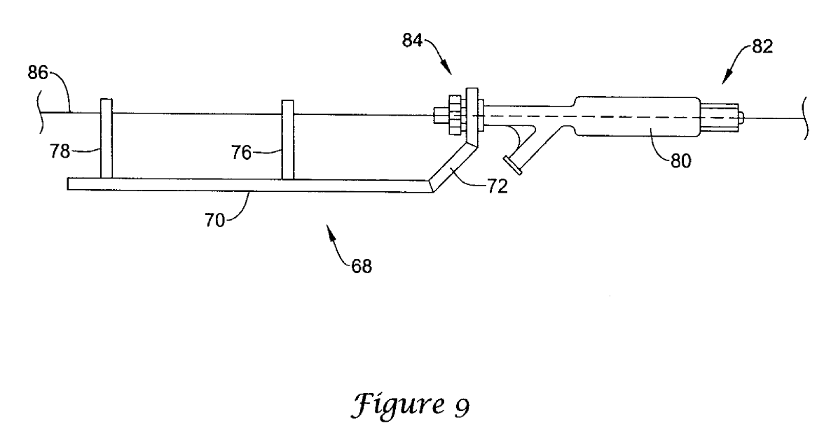

An illustrative but non-limiting use of the clamping apparatus 68 is seen in FIG. 10. An exchange device 88 is shown as a rapid exchange catheter, but may be any other appropriate medical device that may be exchanged over the wire 86. The exchange device 88 has a distal end 90 and a guidewire lumen extending from the distal end 90 to a guidewire port 92. In some instances, the guidewire lumen may extend from the guidewire port 92 to a distal guidewire port (not shown) that may be positioned at or near the distal end 90.

In use, the first clamp 76 may be closed to secure the wire 86 in position. Then, the exchange device 88 may be advanced over the wire 86 such that the wire 86 enters the distal end 90 and exits through the guidewire port 92. At this point, the monorail section of the exchange device 88 may be positioned between the first clamp 76 and the second clamp 78. The exchange device 88 may extend proximally radially apart from the wire 86 (as shown), or the exchange device 88 may remain parallel to the wire 86.

Next, the second clamp 78 may be closed to secure the wire 86 and the first clamp 76 may then be opened to permit the exchange device 88 to continue distally into the hub 80 and then into any appropriate guide catheter or other elongate device to which the hub 80 is secured. To subsequently remove the exchange device 88, the exchange device 88 may be moved proximally until the monorail section thereof is disposed between the first clamp 76 and the second clamp 78. The first clamp 76 may be closed to secure the wire 86, and the second clamp 78 may be opened to permit the exchange device 88 to continue moving proximally.

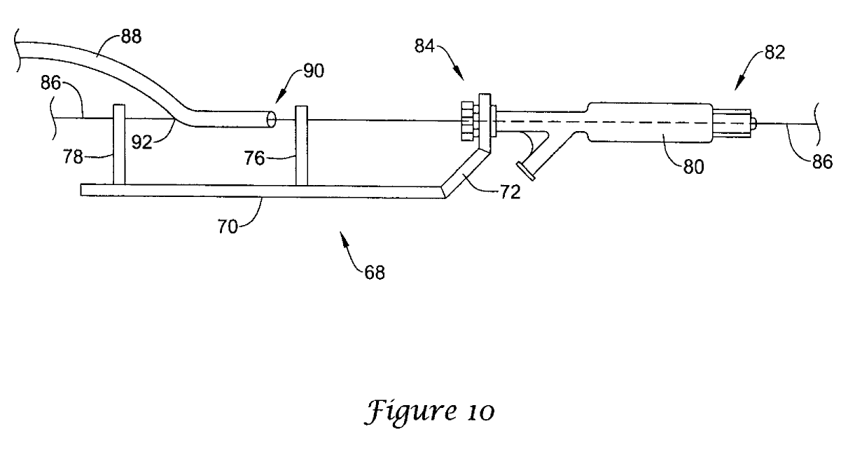

FIGS. 11 through 15 demonstrate illustrative but non-limiting embodiments of the invention in which an apparatus including an embolic protection device includes an anchor to selectively reduce or eliminate movement in a wire such as a filterwire. As noted previously, movement may be defined as axial movement, radial movement, or a combination of both axial and radial movement.

FIG. 11 shows a distal portion of a guide catheter 94 defining a lumen 96. The guide catheter 94 has a distal end 98. An anchor 100 is disposed within the lumen 96, near the distal end 98. The anchor 100 may be configured to be moveable between an open configuration (as illustrated in FIG. 10) and a closed configuration (as will be discussed with respect to FIG. 11). In the closed configuration, the anchor 100 extends a substantial way into the lumen 96 while in the open configuration, the anchor 100 does not extend a substantial way into the lumen 96.

The anchor 100 may be formed of any suitable material and having any suitable dimensions. In some instances, the anchor 100 may have a largely cylindrical shape. In some cases, the anchor 100 may instead include two or more segments that are anchored at one end to the interior of the lumen 96 and that extend proximally within the lumen 96 at the other end thereof.

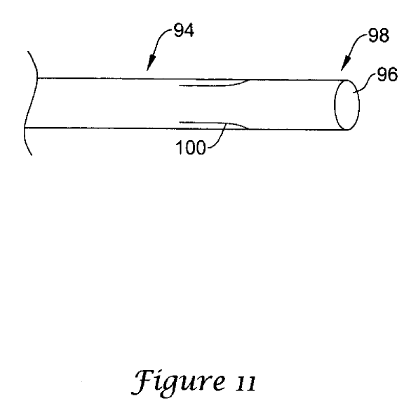

In FIG. 12, the anchor 100 has been moved into its closed configuration in which the anchor 100 extends a substantial distance into the lumen 96. A wire 102 bearing an embolic protection device 36 (as discussed previously) extends distally from the guide catheter 94. A hypotube 104, having a distal end 106, is disposed within the lumen 96 such that the distal end 106 has pushed against the anchor 100, causing the anchor 100 to bend into the lumen 96 such that the anchor 100 contacts the wire 102 and in fact restrains the wire 102 from movement. The hypotube 104 may be formed of any suitable materials.

In some instances, the anchor 100 may be biased into the open configuration. Thus, if it is desired to restrain the wire 102 from movement, the hypotube 104 may be moved distally until the anchor 100 has been moved into the closed configuration. Subsequent proximal movement of the hypotube 104 may permit the anchor 100 to revert to the open configuration and thus permit the wire 102 to move once again.

In some cases, the anchor 100 may be moved into the closed configuration so that another device may be advanced through the guide catheter 94. As the anchor 100 is located close to the distal end 98 of the guide catheter 94, it can be seen that the anchor 100 may be used to constrain movement of the wire 102 while the new device is advanced quite a ways over the wire 102. Only once the new device is proximate the anchor 100 does the anchor 100 need to be moved back into the open configuration.

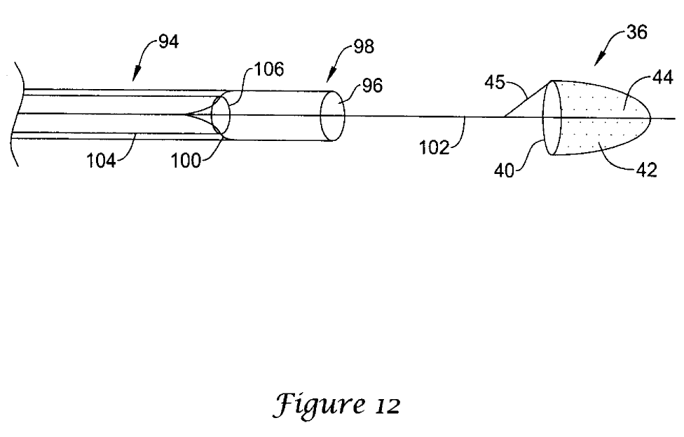

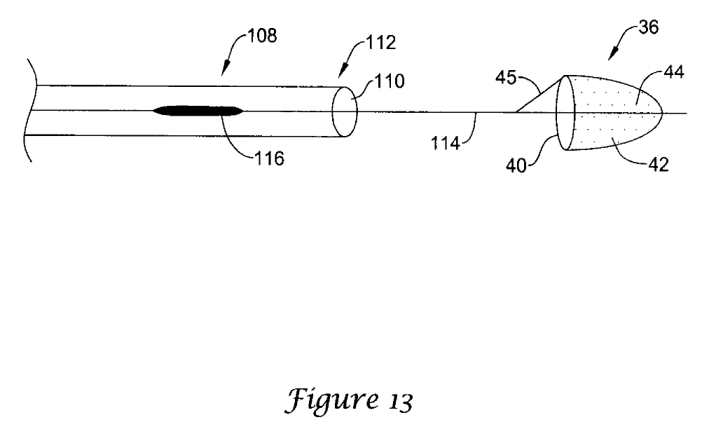

FIG. 13 shows a distal portion of a guide catheter 108 defining a lumen 110. The guide catheter 108 has a distal end 112. A shaft 114 extends through the lumen 110. The shaft 114 bears a distal protection device 36 (as discussed previously) as well as an inflatable balloon 116. The inflatable balloon 116 has an open configuration, in which the inflatable balloon 116 is deflated (as seen in FIG. 13), and a closed configuration, in which the inflatable balloon 116 is inflated (as seen in FIG. 14).

In the closed configuration, the inflatable balloon 116 extends a substantial distance into the lumen 110 such that the inflatable balloon 116 in fact restrains movement of the shaft 114. In the open configuration, the inflatable balloon 116 does not extend substantially into the lumen 110, and other devices may be advanced over the shaft 114 and over the inflatable balloon 116.

The shaft 114 may include an inner lumen (not illustrated) that is in fluid communication with an interior of the inflatable balloon 116 for purposes of inflating and deflating the inflatable balloon 116. Any suitable inflation fluid may be used. In some instances, saline may be an appropriate inflation fluid as saline is essentially incompressible, and is safe for the patient if there are any leaks.

In some cases, the inflatable balloon 116 may be inflated, or moved into the closed configuration, so that another device may be advanced through the guide catheter 108. As the inflatable balloon 116 is positioned close to the distal end 112 of the guide catheter 108, it can be seen that the inflatable balloon 16 may be used to constrain movement of the shaft 114 while the new device is advanced quite a ways over the shaft 114. Only once the new device is proximate the inflatable balloon 116 does the inflatable balloon 116 need to be deflated, or moved back into the open configuration.

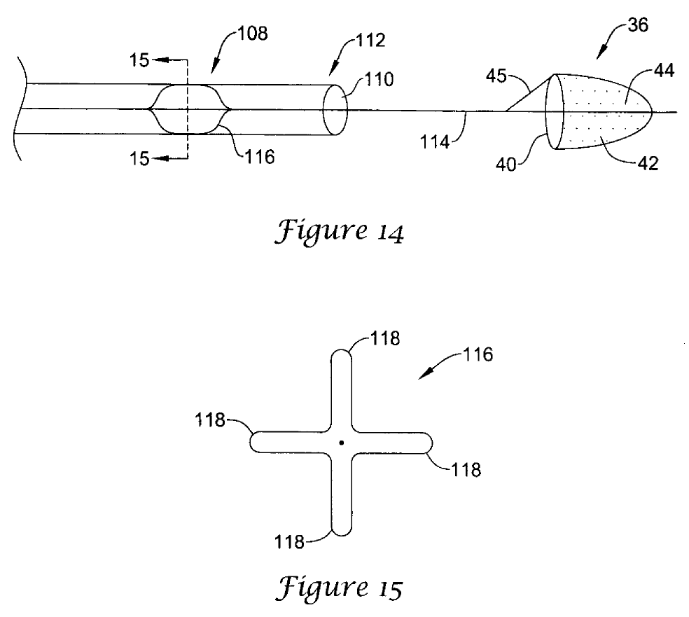

In some instances, there may be a desire to immobilize the shaft 114 while not entirely sealing off the lumen 110 within the guide catheter 108. As shown in FIG. 15, the inflatable balloon 116 may have a profile, when inflated, that includes two or more lobes 118. As illustrated, the inflatable balloon 116 has a total of four equally spaced lobes 118. In other instances, the inflatable balloon 116 may have three, five or more lobes 118 that may be equally spaced or may be unequally spaced. The lobes 118 permit the inflatable balloon 116 to contact the lumen 110, thereby restraining the shaft 114, while still permitting fluid such as contrast fluid to pass through the guide catheter 108.

The devices described herein may include a variety of different materials. These materials may include metals, metal alloys, polymers, metal-polymer composite, and the like, or any other suitable material. Some examples of suitable metals and metal alloys include stainless steel, such as 304V, 304L, and 316LV stainless steel; mild steel; nickel-titanium alloy such as linear-elastic or super-elastic nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), hastelloy, monel 400, inconel 825, or the like; other Co—Cr alloys; platinum enriched stainless steel; or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In addition, the devices described herein may also be doped with or otherwise include a radiopaque material. Radiopaque materials are understood to be materials capable of producing a relatively bright image on a fluoroscopy screen or another imaging technique during a medical procedure. This relatively bright image aids the user of filtering device in determining their location. Some examples of radiopaque materials can include, but are not limited to, gold, platinum, molybdenum, palladium, tantalum, tungsten or tungsten alloy, plastic material loaded with a radiopaque filler, and the like.

The invention should not be considered limited to the particular examples described above, but rather should be understood to cover all aspects of the invention as set out in the attached claims. Various modifications, equivalent processes, as well as numerous structures to which the invention can be applicable will be readily apparent to those of skill in the art upon review of the instant specification

We claim:

1. A hub assembly comprising:
a hub body defining a hub lumen therethrough;
a magnetic column disposed exterior to the hub lumen; and
a wire disposed within the hub lumen.

2. The hub assembly of claim 1, wherein the magnetic column is molded within the hub body.

3. The hub assembly of claim 1, wherein the magnetic column is secured exterior to the hub body.

4. The hub assembly of claim 3, wherein the magnetic column is hingedly attached to the hub body.

5. The hub assembly of claim 3, wherein the hub body comprises an indentation configured to accommodate the magnetic column.

6. The hub assembly of claim 1, wherein the hub body comprises a transparent or translucent plastic material.

7. The hub of claim 6, wherein the hub body comprises polycarbonate.

8. A hub comprising:
a hub body defining a hub lumen therethrough;
a magnetic column disposed at least partially about the hub lumen; and
an introducer sheath removably disposed within the hub lumen;
wherein the introducer sheath reduces a magnetic field within at least a portion of the hub lumen.

9. The hub of claim 8, wherein the introducer sheath comprises a material having a high magnetic permeability.

10. The hub of claim 6, wherein the magnetic column comprises a plurality of semi-circular magnets molded within the hub body.

11. The hub of claim 6, wherein each of the plurality of semi-circular magnets extend around about 180 degrees to about 270 degrees of a circle.

12. The hub of claim 6, further comprising a wire disposed within the hub lumen.

13. The hub of claim 12, wherein the wire is axially movable within the hub lumen when the introducer sheath is disposed within the hub lumen but is not axially movable when the introducer sheath is removed from the hub lumen.

14. A hub assembly comprising:
a hub body having an interior and an exterior, the interior defining a hub lumen;
a wire disposed within the hub lumen; and
a magnetic column disposed about the hub body exterior.

15. The hub assembly of claim 14, wherein the magnetic column is removably secured to the hub body exterior.

16. The hub assembly of claim 14, wherein the magnetic column is hingedly secured to the hub body exterior.

17. The hub assembly of claim 14, wherein the wire comprises a guidewire or a filterwire.

18. A clamping apparatus comprising:
an elongate body having a distal end and a proximal end, the distal end adapted to be secured to a medical hub;
a first clamp secured to the elongate body; and
a second clamp secured to the elongate body, the second clamp spaced apart from the first clamp.

19. The clamping apparatus of claim 18, wherein the medical hub accommodates a guidewire therein, and the first and second clamps are spaced apart a distance sufficient to accommodate a monorail section of a medical device adapted to be advanced over the guidewire.

20. The clamping apparatus of claim 19, wherein the first clamp is movable between an open position in which the first clamp is configured to accommodate the medical device and a closed position in which the first clamp is configured to secure the guidewire.

21. The clamping apparatus of claim 19, wherein the second clamp is movable between an open position in which the second clamp is configured to accommodate the medical device and a closed position in which the second clamp is configured to secure the guidewire.

22. The clamping apparatus of claim 18, wherein the first clamp and the second clamp are mechanically actuated.

23. The clamping apparatus of claim 18, wherein the first clamp and the second clamp are electromagnetically actuated.

24. The clamping apparatus of claim 18, wherein the second clamp is disposed about 10 to about 20 centimeters from the first clamp.

25. A method of advancing a medical device over a wire, the medical device including a monorail section, the method comprising the steps of:
advancing a wire through a medical apparatus, the medical apparatus including a clamping apparatus extending proximally from the medical apparatus, the clamping apparatus comprising a first clamp and a second clamp spaced apart from the first clamp;
securing the first clamp to the wire;
advancing the medical device monorail section over the wire such that the monorail section is disposed between the first clamp and the second clamp;
securing the second clamp to the wire;
releasing the first clamp; and
further advancing the medical device over the wire.

26. The method of claim 25, wherein advancing a wire through a medical apparatus comprises advancing a wire through a guide catheter.

27. The method of claim 25, wherein advancing a wire through a medical apparatus comprises advancing a wire through a hub.

28. An assembly, comprising:
a guide catheter having a distal region, a proximal region and a lumen extending therebetween;
an elongate shaft having a distal portion and a proximal portion, the proximal portion disposed within the lumen, the distal portion extending distally beyond the guide catheter;
an embolic protection device disposed on the distal portion of the elongate shaft; and
an anchor disposed within the distal region of the guide catheter, the anchor having an activated configuration in which the anchor extends considerably into the lumen and an inactivated configuration in which the anchor does not extend considerably into the lumen.

29. The assembly of claim 28, wherein a wire extending through the guide catheter is at least substantially immobilized when the anchor is in the activated configuration.

30. The assembly of claim 28, further comprising a hypotube disposed within the lumen, wherein distal movement of the hypotube shapes the anchor into the activated configuration.

31. The assembly of claim 30, wherein the anchor is biased into the inactivated configuration.

32. The assembly of claim 28, wherein the anchor comprises an inflatable balloon disposed on the proximal portion of the elongate shaft, the inflatable balloon having an inflated configuration in which the inflatable balloon interacts with the lumen to secure the elongate shaft relative thereto.

33. The assembly of claim 32, wherein the inflatable balloon comprises, when inflated, one or more lobes that are configured to interact with the lumen yet permit fluid flow.
